(19) United States
(12) Patent Application Publication
(10) Pub. No.: US 2010/0035994 A1
(43) Pub. Date: Feb. 11, 2010

(54) NOVEL HYDRATE FORM OF O-DESMETHYL VENLAFAXINE SUCCINATE

(76) Inventors: Vinayak G. Gore, Maharashtra (IN); Vikas S. Kulkarni, Maharashtra (IN); Vikas S. Wakchaure, Maharashtra (IN); Mahesh G. Hublikar, Maharashtra (IN); Sneha R. Wavhal, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/376,537

(22) PCT Filed: Aug. 8, 2007
(86) PCT No.: PCT/GB07/50477

§ 371 Date: Jun. 19, 2009

(30) Foreign Application Priority Data
Aug. 8, 2006 (IN) 1256/MUM/2006

(51) Int. Cl. A61K 31/19; C07C 55/10; A61P 25/00; A61P 15/00; A61P 13/00

(52) U.S. Cl. 514/574; 562/590

(57) ABSTRACT

The present invention relates to a novel hydrate form of O-desmethyl venlafaxine succinate. The present invention further relates to processes for the preparation of the novel hydrate form, pharmaceutical compositions comprising it, second medical uses of the novel hydrate form, and methods using it for treating diseases such as generalised anxiety disorder, anxiety, depressive disorder, depression and panic disorder.

### CROSS-REFERENCE TO RELATED APPLICATION(S)

This application is a Section 371 National Stage Application of International No. PCT/GB2007/050477, filed 8 Aug. 2007 and published as WO 2008/017886 A1 on 14 Feb. 2008, which claims priority from the India Application 1256/mum/2006, filed 8 Aug. 2006, the subject matter of which are hereby incorporated by reference in its entirety.

### FIELD OF THE INVENTION

The present invention relates to a novel hydrate form of O-desmethyl venlafaxine (ODV) succinate. The present invention further relates to processes for the preparation of the novel hydrate form, pharmaceutical compositions comprising it, second medical uses of the novel hydrate form, and methods using it for treating diseases such as generalised anxiety disorder, anxiety, depressive disorder, depression and panic disorder.

### BACKGROUND OF THE INVENTION

O-Desmethyl venlafaxine, chemically named 1-[2-(dimethylamino)-1-(4-hydroxyphenyl)ethyl]cyclohexanol, is a major metabolite of venlafaxine. ODV has been shown to inhibit norepinephrine and serotonin uptake. Various patents describe processes for the preparation of ODV free base, which can be converted into desired salts.

For example, U.S. Pat. No. 4,535,186 describes the fumarate salt of ODV. The fumarate salt of ODV, however, has unsuitable physiochemical and permeability characteristics.

The succinate salt of ODV shown below, on the other hand, provides optimal properties for formulation due to its high solubility, permeability and bioavailability.

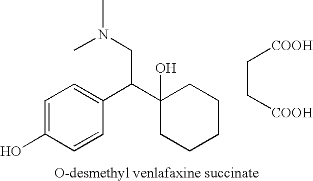

U.S. Pat. No. 6,673,838 indicates that ODV succinate is well absorbed in the gastrointestinal tract. Furthermore, oral administration of ODV succinate, in particular in sustained release formulations, results in a lower incidence of nausea, vomiting, diarrhea, abdominal pain, headache, vasovagal malaise and/or trismus than oral administration of venlafaxine. ODV succinate is known to be effective in treating patients suffering from depression, anxiety, panic disorder etc. The treatment method includes administering to a patient in need thereof an effective amount of ODV succinate or a substantially pure polymorph of ODV succinate or mixtures thereof.

U.S. Pat. No. 6,673,838 describes five polymorphs of ODV succinate (four crystalline polymorphs and one amorphous polymorph) and processes for their preparation. There are two crystalline monohydrate forms (form I and II), one crystalline hydrate form (form III with a water content between hemi- and monohydrate), one crystalline anhydrate form (form IV) and one amorphous form.

U.S. Pat. No. 6,673,838 discloses processes for the preparation of the succinate monohydrate salt of racemic ODV in forms I and II. It describes a process for the preparation of form II from form I, which leads to polymorphic impurities. Similarly, form III is prepared from form I, which again leads to polymorphic impurities. Moreover, form I is unstable and is converted into form III on milling. There are no crystallization conditions described for form III. Form IV can be prepared from a mixture of forms I and II, and the amorphous form can be prepared from form I, II, III or IV or mixtures thereof, which again leads to polymorphic impurities. According to U.S. Pat. No. 6,673,838, the solubility of ODV succinate monohydrate form I is 32 mg/ml.

Preparing a salt or a polymorph of a known compound is a means of altering the physiochemical and biological characteristics of that compound. This is advantageous in dosage form development.

Polymorphism influences every aspect of the solid state properties of a drug and one of the important aspects of polymorphism in pharmaceuticals is the possibility of interconversion among polymorphic forms. Polymorphic forms can differ from each other in properties relevant to the use, efficacy, stability etc. of pharmaceutically important substances.

Solubility is one of the important characteristics of polymorphic forms that can affect their suitability for use as a drug. The present invention provides a novel hydrate form of ODV succinate, which has a better dissolution rate in vivo leading to better bioavailability. The present inventors have studied the novel polymorph at relatively mild conditions and its suitability in dosage form development, e.g. tablet preparation. Moreover, the present invention has the advantage of providing the novel ODV succinate hydrate substantially free from polymorphic impurities, since it is prepared directly form ODV free base.

### OBJECT OF THE INVENTION

It is an object of the present invention to provide a novel hydrate form of O-desmethyl venlafaxine succinate with less hygroscopicity, higher stability, higher solubility and higher bioavailability.

It is a further object of the present invention to provide compositions of the novel hydrate form of ODV succinate.

### SUMMARY OF THE INVENTION

A first aspect of the present invention provides ODV succinate hydrate, having an X-ray diffraction pattern comprising at least three peaks selected from peaks with 2 θ angles of 5.1, 10.2, 13.1, 15.8, 16.6, 17.6, 19.5, 20.3 and 25.7±0.2 degrees. Preferably, the ODV succinate hydrate has an X-ray diffraction pattern comprising at least four, five, six, seven, eight or nine peaks selected from peaks with 2 θ angles of 5.1, 10.2, 13.1, 15.8, 16.6, 17.6, 19.5, 20.3 and 25.7±0.2 degrees. In one embodiment, the ODV succinate hydrate has an X-ray diffraction pattern comprising at least three, four, five, six, seven, eight or nine peaks selected from peaks with 2 θ angles of about 5.05, 10.15, 13.11, 15.79, 16.57, 17.56, 19.52, 20.29 and 25.69. Preferably Cu Kα1 radiation (λ=1.5406 Å) is used to obtain the X-ray diffraction pattern. Preferably the ODV succinate hydrate has a solubility of at least 40 mg/ml, preferably at least 45 mg/ml, preferably at least 50 mg/ml, preferably at least 55 mg/ml, preferably about 55 mg/ml.

The first aspect of the present invention also provides ODV succinate hydrate, having an X-ray diffraction pattern substantially as shown in FIG. 1. Preferably the ODV succinate hydrate has a solubility of at least 40 mg/ml, preferably at least 45 mg/ml, preferably at least 50 mg/ml, preferably at least 55 mg/ml, preferably about 55 mg/ml.

Slight variations in the observed 2 θ angles are expected based on the specific diffractometer used, the analyst and the sample preparation technique. The terms ‘2 θ angles of about’ and ‘an X-ray diffraction pattern substantially as shown’ are to be interpreted accordingly.

The first aspect of the present invention further provides ODV succinate, having a solubility of at least 40 mg/ml, preferably at least 45 mg/ml, preferably at least 50 mg/ml, preferably at least 55 mg/ml, preferably from 50-60 mg/ml, preferably about 55 mg/ml. Preferably the ODV succinate is a hydrate.

The ODV succinate of the present invention can be racemic, stereoisomerically enriched or stereoisomerically pure. Preferably the ODV succinate of the present invention comprises 0.25-0.75 mol water of hydration per mol ODV succinate.

Preferably the ODV succinate of the present invention has a high polymorphic purity and is substantially free of other polymorphic and amorphous forms of ODV succinate. This means that the ODV succinate of the present invention preferably comprises less than 10% of other polymorphic and amorphous forms, preferably less than 5%, preferably less than 2%, preferably less than 1%, preferably less than 0.5%.

Preferably the ODV succinate of the present invention has a high chemical purity. This means that the ODV succinate preferably has a chemical purity of more than 98.5%, preferably more than 99%, preferably more than 99.5%, preferably more than 99.8%, as measured by HPLC.

The ODV succinate of the present invention can be used to advantage in the preparation of pharmaceutical compositions, because the novel ODV succinate form of ODV succinate has a better dissolution rate in vivo and therefore a better bioavailability.

The ODV succinate of the present invention can be used as a medicament, for example, for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

A second aspect of the present invention provides a process of preparing the ODV succinate hydrate of the present invention, comprising the steps of:
(a) forming a suspension of ODV and succinic acid in cyclohexane and water;
(b) heating the suspension;
(c) cooling the suspension; and
(d) filtering the suspension to isolate the ODV succinate hydrate.

Preferably step (a) is performed by adding water to a mixture, for example a suspension, of O-desmethyl venlafaxine and succinic acid in cyclohexane to form the suspension.

The second aspect of the present invention also provides a process of preparing the ODV succinate hydrate of the present invention, comprising the steps of:
(a) providing a mixture of ODV, succinic acid, N,N-dimethylformamide, acetone and water;
(b) heating the mixture;
(c) cooling the mixture; and
(d) filtering the mixture to isolate the ODV succinate hydrate.

Preferably step (a) is performed by adding an aqueous solution of succinic acid to a mixture of ODV, N,N-dimethylformamide and acetone. Alternatively step (a) may be performed by providing a mixture of N,N-dimethylformamide and acetone, and consecutively adding ODV, succinic acid and water.

In both processes, the heating step (b) is preferably carried out in a temperature range of 60° C. to 70° C., preferably at a temperature of about 68° C.

In both processes, the cooling temperature in step (c) is preferably in the range of 20° C. to 30° C., preferably about 25° C.

A third aspect of the present invention provides a pharmaceutical composition comprising the ODV succinate of the present invention and a pharmaceutically acceptable excipient, carrier or diluent.

Preferably the pharmaceutical composition is suitable for oral or parenteral administration. Preferably the pharmaceutical composition is in the form of a tablet, capsule, syrup, suspension or elixir for oral administration or in the form of a sterile solution or suspension for parenteral administration. Tablets can be prepared by conventional techniques, including direct compression, wet granulation and dry granulation. Capsules are generally formed from a gelatine material and contain a conventionally prepared granulate of excipients and ODV succinate of the present invention. Preferably, the dosage form is for oral administration, preferably in the form of a tablet. The pharmaceutical composition may be for immediate, extended or sustained release.

Preferably the pharmaceutical composition is in unit dosage form comprising the ODV succinate in an amount of from 1 mg to 1000 mg, preferably from 10 mg to 750 mg, preferably from 50 mg to 500 mg, as measured by the free base equivalent. The unit dosage form can be administered once, twice, or three times daily.

Preferably the pharmaceutical composition is suitable for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

A fourth aspect of the present invention provides a method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of the ODV succinate of the present invention to a patient in need thereof. Preferably the patient is a mammal, preferably a human. Preferably the amount of the ODV succinate administered is from 0.1 mg to 50 mg per kg per day, preferably from 0.1 mg to 25 mg per kg per day, preferably from 0.2 mg to 10 mg per kg per day.

A fifth aspect of the present invention provides a use of the ODV succinate of the present invention for the manufacture of a medicament for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

The ODV succinate of the present invention can also be useful as precursor to other novel or known polymorphic forms of ODV succinate that may be useful in the preparation of pharmaceutical products.

### BRIEF DESCRIPTION OF THE DRAWINGS

FIG. 1 shows the XRPD (using Cu Kα1 radiation, λ=1.5406 Å) of the novel ODV succinate hydrate form of the present invention.

FIG. 2 shows the DSC of the novel ODV succinate hydrate form of the present invention.

FIG. 3 shows the TGA of the novel ODV succinate hydrate form of the present invention.

### DETAILED DESCRIPTION OF THE INVENTION

As outlined above, the present invention provides a novel hydrate form of O-desmethyl venlafaxine succinate with a characteristic XRD spectrum having major peaks with 2 θ values at about 5.05, 10.15, 13.11, 15.79, 16.57, 17.56, 19.52, 20.29 and 25.69.

The present invention also provides a process for the preparation of the novel hydrate form, comprising the steps of:
(a) adding water to a mixture of O-desmethyl venlafaxine and succinic acid in cyclohexane to form a suspension;
(b) heating the suspension; and
(c) filtering the suspension after cooling to isolate the novel hydrate form.

The present invention further provides a process for the preparation of the novel hydrate form, comprising the steps of:
(a) adding an aqueous solution of succinic acid to a mixture of O-desmethyl venlafaxine, N,N-dimethylformamide and acetone;
(b) heating the mixture; and
(c) filtering the mixture after cooling to isolate the novel hydrate form.

Thus, the present invention provides a novel hydrate form of ODV succinate salt and processes for its preparation. Succinic acid salts of ODV exist as enantiomers and the present invention includes racemic mixtures as well as stereoisomerically pure forms of the same. The term ‘ODV succinate’ as used herein refers to racemic mixtures and stereoisomerically pure forms of ODV succinate, unless otherwise indicated. The term ‘stereoisomerically pure’ refers to compounds, which are comprised of a greater proportion of the desired isomer than of the optical antipode. A stereoisomerically pure compound is generally made up of at least 90% of the desired isomer based upon 100% total weight of ODV succinate salt.

The present invention provides a novel hydrate form of ODV succinate, which is a crystalline hydrate salt. The novel hydrate form of ODV succinate of the present invention has a solubility of 55 mg/ml.

The present invention also provides two processes for the preparation of the novel hydrate form of ODV succinate. The processes of the present invention are capable of providing the novel hydrate form of ODV succinate in consistent chemical and polymorphic purity irrespective of the scale of preparation. The novel hydrate form of ODV succinate can be prepared in batches of 10 g, 50 g, 100 g, 1 kg, 5 kg, 10 kg, 50 kg or more.

The present invention further provides a pharmaceutical composition comprising the novel hydrate form of ODV succinate and a pharmaceutically acceptable excipient, carrier or diluent.

Finally the present invention provides second medical uses of the novel hydrate form of ODV succinate and methods of treating patients suffering from depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine and alcohol addiction, sexual dysfunction, borderline personality disorder, chronic fatigue syndrome, urinary incontinence and Parkinson's disease, the methods comprising providing to a patient an effective amount of the novel hydrate form of ODV succinate.

Details of the invention, its objects and advantages are explained hereunder in greater detail in relation to non-limiting exemplary illustrations.

### EXAMPLES

### Example 1

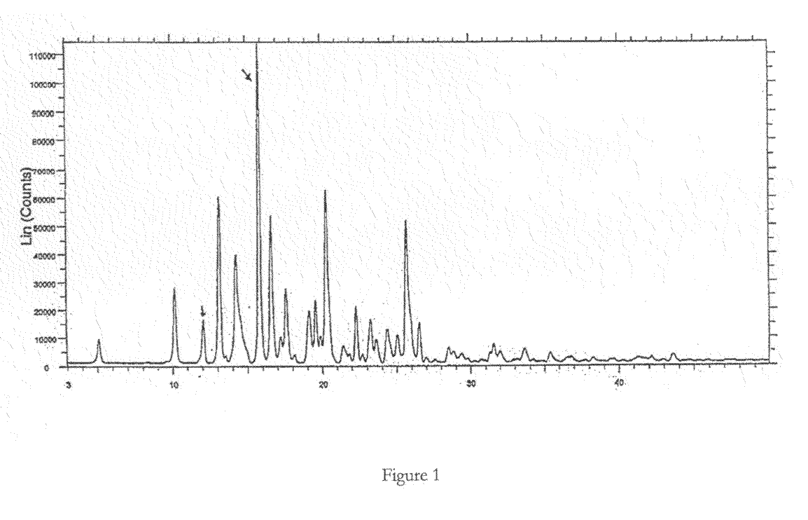

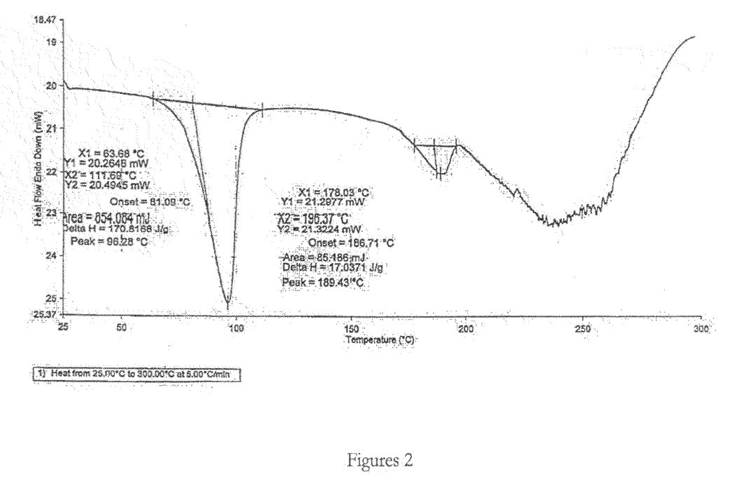

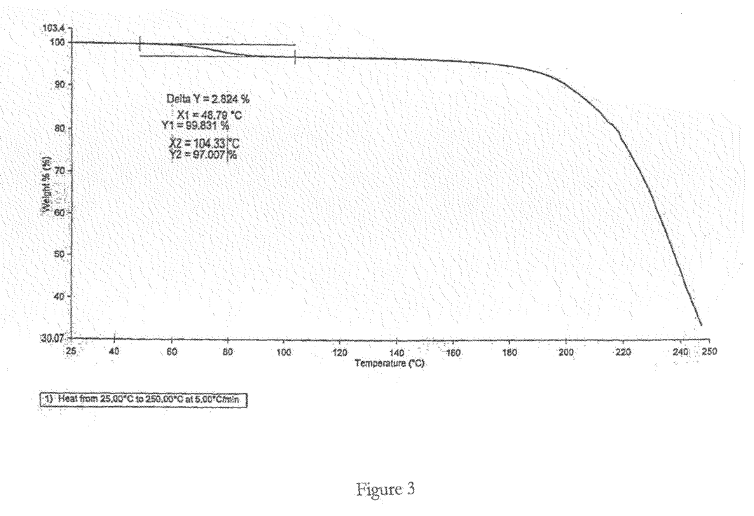

ODV and succinic acid were charged to a reaction flask containing cyclohexane. Water was added to the above mixture. The resulting suspension was heated at 68° C. for two hours under stirring. The reaction mixture was allowed to cool to 25° C. and then filtered. The solid product was dried at 60° C. under vacuum until a constant weight was obtained. The ¹H-NMR indicated formation of ODV succinate. The TGA, shown in FIG. 3, indicated that the ODV succinate salt formed was a hydrate. The XRPD and DSC analysis data, shown in FIGS. 1 and 2 respectively, confirmed that the product obtained was the novel ODV succinate hydrate form of the present invention.

### Example 2

ODV was charged to a reaction flask containing a mixture of N,N-dimethylformamide and acetone. To this stirred mixture, succinic acid was added, followed by water. The resulting mixture was heated at 68° C. for around 90 minutes. The reaction mixture was cooled to 25° C. and then filtered. The solid product was dried at 60° C. under vacuum until a constant weight was obtained. The ¹H-NMR indicated formation of ODV succinate. The TGA indicated that the ODV succinate salt formed was a hydrate. The XRPD and DSC analysis data confirmed that the product obtained was the novel ODV succinate hydrate form of the present invention and that it was identical with that obtained by following example 1.

It will be understood that the present invention has been described above by way of example only. The examples are not intended to limit the scope of the invention. Various modifications and embodiments can be made without departing from the scope and spirit of the invention, which is defined by the following claims only.

What is claimed is:

1. O-Desmethyl venlafaxine succinate hydrate, having an X-ray diffraction pattern comprising at least three peaks selected from peaks with 2 θ angles of about 5.1, 10.2, 13.1, 15.8, 16.6, 17.6, 19.5, 20.3 and 25.7±0.2 degrees.

2. The O-desmethyl venlafaxine succinate hydrate as claimed in claim 1:
(a) having a solubility of at least 40 mg/ml; and/or
(b) comprising less than 10% of O-desmethyl venlafaxine succinate in other polymorphic or amorphous forms; and/or
(c) having a chemical purity of more than 98.5% as measured by HPLC; and/or
(d) for use as a medicament; and/or
(e) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

3. O-Desmethyl venlafaxine succinate hydrate, having an X-ray diffraction pattern substantially as shown in FIG. 1:

4. The O-desmethyl venlafaxine succinate hydrate as claimed in claim 3:
(a) having a solubility of at least 40 mg/ml; and/or
(b) comprising less than 10% of O-desmethyl venlafaxine succinate in other polymorphic or amorphous forms; and/or
(c) having a chemical purity of more than 98.5% as measured by HPLC; and/or
(d) for use as a medicament; and/or
(e) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

5. O-Desmethyl venlafaxine succinate, having a solubility of at least 40 mg/ml.

6. The O-desmethyl venlafaxine succinate hydrate as claimed in claim 5:
(a) comprising less than 10% of O-desmethyl venlafaxine succinate in other polymorphic or amorphous forms; and/or
(b) having a chemical purity of more than 98.5% as measured by HPLC; and/or
(c) for use as a medicament; and/or
(d) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

7. A process of preparing the O-desmethyl venlafaxine succinate hydrate as claimed in claim 1, comprising the steps of:
(a) forming a suspension of O-desmethyl venlafaxine and succinic acid in cyclohexane and water;
(b) heating the suspension;
(c) cooling the suspension; and
(d) filtering the suspension to isolate the O-desmethyl venlafaxine succinate hydrate.

8. The process as claimed in claim 7, wherein:
(a) step (a) is performed by adding water to a mixture of O-desmethyl venlafaxine and succinic acid in cyclohexane to form the suspension; and/or
(b) the heating step (b) is carried out in a temperature range of 60° C. to 70° C.; and/or
(c) the heating step (b) is carried out at a temperature of about 68° C.; and/or
(d) the cooling temperature in step (c) is in the range of 20° C. to 30° C.; and/or
(e) the cooling temperature in step (c) is about 25° C.

9. A process of preparing the O-desmethyl venlafaxine succinate hydrate as claimed in claim 1, comprising the steps of:
(a) providing a mixture of O-desmethyl venlafaxine, succinic acid, N,N-dimethylformamide, acetone and water;
(b) heating the mixture;
(c) cooling the mixture; and
(d) filtering the mixture to isolate the O-desmethyl venlafaxine succinate hydrate.

10. The process as claimed in claim 9, wherein:
(a) step (a) is performed by adding an aqueous solution of succinic acid to a mixture of O-desmethyl venlafaxine, N,N-dimethylformamide and acetone; and/or
(b) the heating step (b) is carried out in a temperature range of 60° C. to 70° C.; and/or
(c) the heating step (b) is carried out at a temperature of about 68° C.; and/or
(d) the cooling temperature in step (c) is in the range of 20° C. to 30° C.; and/or
(e) the cooling temperature in step (c) is about 25° C.

11. A pharmaceutical composition comprising the O-desmethyl venlafaxine succinate as claimed in claim 1 and a pharmaceutically acceptable excipient, carrier or diluent.

12. The pharmaceutical composition as claimed in claim 11, wherein the composition is:
(a) for oral or parenteral administration; and/or
(b) in the form of a tablet, capsule, syrup, suspension or elixir for oral administration or in the form of a sterile solution or suspension for parenteral administration; and/or
(c) in unit dosage form comprising the O-desmethyl venlafaxine succinate in an amount of from 1 mg to 1000 mg, as measured by the free base equivalent; and/or
(d) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

13. A pharmaceutical composition comprising the O-desmethyl venlafaxine succinate as claimed in claim 3 and a pharmaceutically acceptable excipient, carrier or diluent.

14. The pharmaceutical composition as claimed in claim 13, wherein the composition is:
(a) for oral or parenteral administration; and/or
(b) in the form of a tablet, capsule, syrup, suspension or elixir for oral administration or in the form of a sterile solution or suspension for parenteral administration; and/or
(c) in unit dosage form comprising the O-desmethyl venlafaxine succinate in an amount of from 1 mg to 1000 mg, as measured by the free base equivalent; and/or
(d) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

15. A pharmaceutical composition comprising the O-desmethyl venlafaxine succinate as claimed in claim 5 and a pharmaceutically acceptable excipient, carrier or diluent.

16. The pharmaceutical composition as claimed in claim 15, wherein the composition is:
(a) for oral or parenteral administration; and/or
(b) in the form of a tablet, capsule, syrup, suspension or elixir for oral administration or in the form of a sterile solution or suspension for parenteral administration; and/or
(c) in unit dosage form comprising the O-desmethyl venlafaxine succinate in an amount of from 1 mg to 1000 mg, as measured by the free base equivalent; and/or
(d) for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease.

17. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of the O-desmethyl venlafaxine succinate as claimed in claim 1 to a patient in need thereof.

18. The method as claimed in claim 17, wherein:
(a) the patient is a mammal; and/or
(b) the patient is a human; and/or
(c) the amount of the O-desmethyl venlafaxine succinate administered is from 0.1 mg to 50 mg per kg per day.

19. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of the O-desmethyl venlafaxine succinate as claimed in claim 3 to a patient in need thereof.

20. The method as claimed in claim 19, wherein:
(a) the patient is a mammal; and/or
(b) the patient is a human; and/or
(c) the amount of the O-desmethyl venlafaxine succinate administered is from 0.1 mg to 50 mg per kg per day.

21. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence, or Parkinson's disease, comprising administering a therapeutically or prophylactically effective amount of the O-desmethyl venlafaxine succinate as claimed in claim 5 to a patient in need thereof.

22. The method as claimed in claim 21, wherein:
(a) the patient is a mammal; and/or
(b) the patient is a human; and/or
(c) the amount of the O-desmethyl venlafaxine succinate administered is from 0.1 mg to 50 mg per kg per day.
